(19) United States
(12) Patent Application Publication
(10) Pub. No.: US 2025/0352593 A1
(43) Pub. Date: Nov. 20, 2025

(54) USE OF A MICROALGAE EXTRACT, ALONE OR IN COMBINATION, FOR IMPROVING COGNITIVE ABILITIES

(71) Applicant: MICROPHYT, BAILLARGUES (FR)

(72) Inventors: Jonathan MAURY, BAILLARGUES (FR); Remi PRADELLES, BAILLARGUES (FR); Ilya ZHIVKOVICH, MILFORD, PA (US)

(21) Appl. No.: 18/874,477

(22) PCT Filed: Jun. 14, 2023
(86) PCT No.: PCT/EP2023/066010

§ 371 Date: Dec. 12, 2024

(30) Foreign Application Priority Data
Jun. 16, 2022 (EP) 22305879.3

(51) Int. Cl. A61K 36/03; A61K 9/00; A61K 36/77; A61K 47/14; A61K 47/55; A61P 25/00

(52) U.S. Cl. CPC A61K 36/03; A61K 9/0053; A61K 36/77; A61K 47/14; A61K 47/551; A61P 25/00; A61K 2236/30

(57) ABSTRACT

The invention relates to the use of an extract of Phaeodactylum tricornutum alone or in combination with an extract chosen from a guarana (Paullinia cupana) extract, a ginseng (Panax ginseng) extract, a Ginkgo biloba extract, a Nelumbo nucifera extract, a Centella asiatica extract, a Piper nigrum extract, a Bacopa monnieri extract or any combination thereof and/or a compound chosen from arginine, creatine, caffeine, theanine, vitamin C, vitamin E, vitamin B, theophylline, paraxanthine, theobromine, rosmarinic acid, ellagic acid, cinnamaldehyde or any combination thereof, for maintaining and/or increasing data processing speed and/or concentration in healthy mammals. The combination of the extract of P. tricornutum and one of these extracts or one of these compounds is also described. The invention also relates to a composition comprising the combination according to the invention.

The invention relates to the use of an extract of the microalga Phaeodactylum tricornutum alone or in combination for maintaining and/or increasing data processing speed and/or concentration in healthy mammals.

Cognitive functions correspond to the brain functions responsible for the acquisition, processing, memory and use of all data from the environment. They enable reasoning, adaptation, concentration but also communication. Certain periods of life, certain habits or certain practices require individuals to have increased attention, memory and speed of acquisition, and also increased speed in sorting and selecting relevant data from memory. Specifically, cognitive processes may be broken down into a learning phase (data storage), a data processing phase within the memory (speed and quality of analysis) in relation to a situation requiring a decision, and then the motor response enabling the decision to be implemented, making it real and effective. The data processing phase is crucial: over and above the storage capacity and quality of an individual's memory, it is their ability to process memorized data, both in terms of quality (accuracy) and speed, that enables them to adapt optimally to their social, professional and private environment. A typical case is that of students, in learning conditions and particularly in exam conditions, a period during which their cognitive functions are very much in demand. Another example is represented by video game players, of whom it is known that some of their cognitive functions in particular, such as speed of decision-making and adaptation to a changing environment, are called upon during play. These populations of individuals do not suffer from cognitive impairment, or even mild cognitive impairment, defined as impaired cognitive capacities without dementia. They are not an elderly population in cognitive decline. Nor are they individuals who have undergone any prenatal stress that might explain an acquired cognitive deficit. Nor, a fortiori, are they individuals suffering from a pathology associated with cognitive impairment, such as Alzheimer's disease.

Drug treatments based on molecules such as donepezil, rivastigmine, galantamine, memantine, donanemab, TTP488 or aducanumab are already known and have demonstrated their effect on the treatment or prevention of cognitive deficit or decline. Food supplements, notably caffeine- and vitamin-based, are also available on the market to address this deficit. However, there is a need in the nutraceutical field for alternative natural ingredients that do not prevent or treat a cognitive deficit or decline, and that do not consist of a pharmaceutical treatment. The issue here is a need for healthy individuals, young adults or adults without deficits, and not elderly.

This is precisely what the present invention proposes to achieve, by proposing a completely natural Phaeodactylum tricornutum extract which is active on specific components of cognitive functions. Moreover, as will be shown in the present patent application, the Applicant has surprisingly demonstrated a synergistic effect of an extract of P. tricornutum with known extracts or compounds such as vitamins C, E, B6, B9, B12, guarana (Paullinia cupana), extracts of Nelumbo nucifera, Centella asiatica, Piper nigrum, ginseng (Panax ginseng), Ginkgo biloba (G. biloba), bacopa (Bacopa monnieri), creatine, arginine, caffeine, theanine, theophylline, paraxanthine, theobromine, phenolic compounds such as rosmarinic acid, ellagic acid and cinnamaldehyde.

One of the advantages of the extract of P. tricornutum according to the invention is that it is a completely natural extract that is not toxic to humans and does not cause allergies. It can be obtained by growing the microalga under controlled conditions in a photobioreactor, and can therefore be readily produced on an industrial scale.

The microalga P. tricornutum is a microalga belonging to the Phaeodactylum genus and the Phaeodactylaceae family. It is a microalga with three distinct morphotypes (fusiform, oval, triradiate) found in many oceanic regions of the world, including northern Europe, Oceania and the Atlantic.

The Applicant's patent application FR3092968A1 describes extracts of microalgae of the species Phaeodactylum tricornutum and Tisochrysis lutea for preparing a dietary supplement for preventing or combating age-related cognitive disorders or cognitive disorders in children or young adults who have undergone prenatal stress. However, the aim of the present patent application is to prevent a decline in learning deficits, notably to improve spatial working memory and long-term contextual memory. The extracts described make it possible to compensate for behavioural alterations linked to biochemical alterations (increase in oxidative stress and induction of neuroinflammatory processes involving the release of interleukins) which are observed in practice in populations of individuals with non-pathological cognitive disorders and in particular in young children or adults who have undergone prenatal stress, or in elderly subjects, which is not the case for the population targeted in the context of the present invention. Nothing in this patent application FR3092968A1 discloses an effect of an extract of P. tricornutum for maintaining and/or increasing data processing speed and/or concentration within populations of individuals with no cognitive deficits. A fortiori, the patent application does not disclose the use of this extract in combination with another extract or compound to further increase its effects. It is noted that, for the purposes of the invention, a clear distinction is made between an effect on an individual's memory and an effect on the speed of processing memorized data.

Similarly, an effect on an individual's concentration is distinct from their ability to memorise data.

Thus, nothing in said patent application FR3092968A1 can encourage a person skilled in the art to use one of the microalgal extracts described to hope to maintain and/or increase data processing speed and/or concentration within a population otherwise distinct from that described in said patent application, all the less so in combination with another compound or extract to potentiate the effects thereof.

Moreover, patent application WO 2019/193596 A1 describes a method for reducing the accumulation of lipids in cells, aimed at the prevention or treatment of liver or kidney dysfunction. Said method makes use of an extract of the same microalga P. tricornutum. However, the use of the present invention is entirely distinct therefrom. Moreover, the method disclosed herein is a therapeutic treatment method, which is aimed at a particular population of individuals. Thus, there is nothing in said document to encourage a person skilled in the art to use this extract of P. tricornutum to implement the invention described in the present patent application.

Thus, to the best of the Applicant's knowledge, no prior art discloses the use described in the present patent application. Furthermore, no prior art describes the combination as such of an extract of P. tricornutum with an extract chosen from a guarana (Paullinia cupana) extract, a ginseng (Panax ginseng) extract, a Ginkgo biloba extract, a Nelumbo nucifera extract, a Centella asiatica extract, a Piper nigrum extract, a bacopa (Bacopa monnieri) extract or with a compound chosen from creatine, arginine, caffeine, theanine, vitamin C, vitamin E, vitamin B, theophylline, paraxanthine, theobromine, rosmarinic acid, ellagic acid and cinnamaldehyde.

Thus, a first subject relates to the use of an extract of Phaeodactylum tricornutum alone or in combination for maintaining and/or increasing data processing speed and/or concentration in healthy mammals, in particular healthy individuals not showing any cognitive decline and/or disorder. A second subject relates to the combination of an extract of P. tricornutum with an extract chosen from a guarana (Paullinia cupana) extract, a ginseng (Panax ginseng) extract, a Ginkgo biloba extract, a Nelumbo nucifera extract, a Centella asiatica extract, a Piper nigrum extract, a B. monnieri extract or a compound chosen from creatine, arginine, caffeine, theanine, vitamin C, vitamin E, vitamin B, theophylline, paraxanthine, theobromine, rosmarinic acid, ellagic acid, cinnamaldehyde, advantageously with an extract chosen from a guarana (Paullinia cupana) extract, a Nelumbo nucifera extract, a Centella asiatica extract, a Piper nigrum extract, or any combination thereof and/or a compound chosen from creatine, arginine, caffeine, theophylline, paraxanthine, theobromine, rosmarinic acid, ellagic acid, cinnamaldehyde or any combination thereof. A third subject relates to a composition, advantageously for use as a food supplement, comprising the combination according to the invention.

A first subject of the invention thus relates to the use of an extract of Phaeodactylum tricornutum alone or in combination with an extract chosen from a guarana (P. cupana) extract, a ginseng (P. ginseng) extract, a G. biloba extract, an N. nucifera extract, a C. asiatica extract, a P. nigrum extract, a B. monnieri or any combination thereof and/or a compound chosen from creatine, arginine, caffeine, theanine, vitamin C, vitamin E, vitamin B, theophylline, paraxanthine, theobromine, rosmarinic acid, ellagic acid, cinnamaldehyde or any combination thereof, advantageously with an extract chosen from a guarana (Paullinia cupana) extract, a Nelumbo nucifera extract, a Centella asiatica extract, a Piper nigrum extract, or any combination thereof and/or a compound chosen from creatine, arginine, caffeine, theophylline, paraxanthine, theobromine, rosmarinic acid, ellagic acid, cinnamaldehyde or any combination thereof, for maintaining and/or increasing data processing speed and/or concentration in healthy mammals, advantageously in healthy individuals not showing any cognitive decline and/or disorder. Advantageously, this is a non-therapeutic use, in particular a nutraceutical use.

The term “mammal” means herein a human or an animal, advantageously a domestic animal, even more advantageously a dog or cat. Very advantageously, it is a human. The term “healthy individuals” means individuals who do not show any cognitive decline and/or disorder. This population therefore excludes elderly individuals, in particular elderly humans, i.e. over 60 years old, advantageously over 70 years old, individuals suffering from depression, including chronic depression, but also mammals showing a cognitive deficit following a stroke, cancer, diabetes, physical trauma to the brain, chronic kidney disease, or Alzheimer's disease, individuals showing any cognitive behavioural modification linked to oxidative stress or a neuro-inflammatory state, and also children or young adults who have undergone prenatal stress inducing non-pathological disorders, such as hyperactivity, attention and memory deficit, language delay and anxious behaviour.

The term “non-therapeutic use” means a non-pharmaceutical use, which is therefore not intended as a therapeutic treatment within the understanding of a specialist in the field. It is aimed at the population of individuals qualified as healthy as defined above.

The term “vitamin B” means vitamin B6, B9, B12 or any combination thereof. The term “oral route” also means ingestion of the extract of P. tricornutum alone or in combination, or of a composition comprising it.

“Increasing data processing speed in healthy mammals” means reducing the time required for a healthy mammal to analyse information from an external stimulus chosen from a visual stimulus, an auditory stimulus, an olfactory stimulus or a gustatory stimulus in order to make a decision. In an advantageous embodiment, this involves a reduction in reaction time in response to a visual stimulus, preferentially in healthy individuals.

Thus, advantageously, the reduction in said reaction time is a significant reduction in the response time of at least 9 ms, advantageously of at least 30 ms, very advantageously of at least 60 ms measured within a population of healthy individuals to whom a combination of guarana and P. tricornutum extract according to the invention has been administered, compared with the reaction time measured within the same population receiving a placebo ingredient.

The measurement is advantageously performed within the framework of a clinical study using the Sternberg test (Sternberg S., 1969) which consists in presenting participants with visual stimuli, one by one, identifying them as present or absent in sequences of 3, 6, 9, 12, 15 or 18 seconds apart: the number of visual stimuli (data to be processed) is increased over short time intervals and the level of difficulty is thus increasing. To avoid repetition, participants are instructed to count backwards by threes and fours to a specific random number, until they see a red light appear on the computer screen. This test specifically measures accuracy and reaction time.

In an advantageous embodiment, the reduction in reaction time is measured within a population composed of 51 healthy men and 10 healthy women, qualified as experienced players, with an average age of 17.7 to 25.7 years, a weight of 60 to 86 kg, and a body mass index ranging from 20.6 to 27.8 kg/m². Advantageously, the individuals did not consume stimulants, dietary supplements comprising theophylline, paraxanthine, theobromine, vitamins, creatine, caffeine, theanine, or any other substance or extract containing any of the abovementioned compounds alone or in combination, nor did they consume a guarana extract or a ginseng extract or a G. biloba extract, or a P. tricornutum extract, or foods rich in arginine or nitrates, during the 2 weeks prior to the start of the clinical study. Even more advantageously, the combination administered is a combination of 500 mg of a guarana extract and a dose of 436 mg/day or 872 mg/day of a composition comprising the extract of P. tricornutum according to the invention, tocopherol and an MCT oil as described in the remainder of the present patent application, under the conditions as described in Example 3. Very advantageously, the extract of P. tricornutum included in the composition is the extract as prepared in Example 1 and the guarana extract is the extract of guarana seeds as prepared in Example 2.

In an alternative embodiment, “increasing data processing speed in healthy individuals” means increasing the protein expression of a synaptotagmin chosen from synaptotagmin 1 (SYT1), SYT2, SYT3, SYT4, SYT5, SYT6, SYT7, SYT8, SYT9, SYT10, SYT11, SYT12, SYT13, SYT14, SYT15, SYT16, SYT17, in a culture of neurons in the presence of the extract of P. tricornutum according to the invention.

Advantageously, the increase in protein expression is at least 2%, advantageously at least 4% in the presence of the extract according to the invention compared with a control without extract. Advantageously, this is an increase in protein expression of synaptotagmin measured in the presence of the extract of P. tricornutum as prepared according to Example 1.

In another alternative embodiment, “increasing data processing speed in healthy individuals” means increasing the density of the neuronal network at neuronal cell level by increasing the length and/or dendricity of neuronal extensions in healthy individuals. The term “dendricity” refers to the formation of dendrites (extensions of the cell body of neurons). Specifically, it is well known that all the cognitive components, and in particular the speed at which individuals process data, are closely linked to neuronal cell density.

Thus, advantageously, the extract of P. tricornutum according to the invention increases by at least 2.5%, preferentially by at least 5%, the length of neuronal extensions in vitro, in a culture of neurons, compared with the length of said extensions measured without the extract. Advantageously, the increase in neuronal extensions is measured in a neurone culture in the presence of an amount of extract according to the invention by weight relative to the total weight of the culture medium and the extract of between 0.0005% and 1.5%, advantageously between 0.001% and 0.5%. Advantageously, the extract of P. tricornutum is as prepared in Example 1. Very advantageously, the length of the neurons is measured after labelling the neurons with an anti-β-tubulin antibody and then visualization using a secondary antibody coupled to a fluorochrome using photographs taken by an automatic microscope, in particular under conditions as described in Example 5a).

Alternatively, the increase in data processing speed in healthy individuals is assessed by measuring neuronal dendricity in the same in vitro neuron culture model as that described above, in the presence of an amount of extract according to the invention by weight relative to the total weight of the culture medium and the extract of between 0.0005% and 1.5%, advantageously between 0.001% and 0.5%. Advantageously, the extract of P. tricornutum is as prepared in Example 1. Very advantageously, neuronal dendricity is assessed by measuring the formation of neuronal bifurcations, by microscopy as detailed above.

In yet another alternative embodiment of the invention, “increasing data processing speed in healthy individuals” means increasing in vitro the rate of calcium mobilization at synapses during synaptic transmission (transmission of nerve impulses between two neurons). Specifically, calcium channels are essential for the transmission of nerve impulses; when they open as a result of the depolarization of synaptic buttons following an action potential, they allow a flow of calcium ions into the synaptic buttons, triggering the fusion of synaptic vesicles, which release neurotransmitters such as acetylcholine. Thus, an in vitro measurement of the level of calcium mobilized in a culture of neurons in the presence of the extract according to the invention plausibly makes it possible to reflect an increase in data processing speed. In an advantageous embodiment, the measurement of the level of mobilized calcium is performed using a fluorescent probe which binds to calcium under the effect of its release following activation of neuronal receptors, in particular under conditions such as those described in Example 5b). Advantageously, the increase in data processing speed by the extract according to the invention is measured in a neuron culture in the presence of an amount of between 0.0005% and 1.5%, advantageously between 0.001% and 0.5%. Advantageously, the extract according to the invention is the extract of P. tricornutum as prepared in Example 1.

On the other hand, “maintaining data processing speed in healthy mammals” means maintaining the reaction time required by a healthy mammal, advantageously a healthy individual, in response to an increasing number of stimuli (data to be processed). In a preferential embodiment, this means maintaining the reaction time measured in the presence of the combination of guarana and the extract of P. tricornutum according to the invention, compared with the reaction time measured within the population having received the placebo ingredient. Advantageously, “maintaining data processing speed in healthy individuals” means maintaining the reaction time measured in the presence of the combination of guarana and the extract of P. tricornutum when the number of visual stimuli (data to be processed d) over a given time is increased by 4 to 6. Even more advantageously, this is the measurement of the reaction time when measured in the clinical study described previously. Even more advantageously, the combination administered is a combination of 500 mg of a guarana extract and a dose of 436 mg/day or 872 mg/day of a composition comprising the extract of P. tricornutum according to the invention, tocopherol and an MCT oil as described in the remainder of the present patent application, under the conditions as described in Example 3. Very advantageously, the extract of P. tricornutum included in the composition is the extract as prepared in Example 1 and the guarana extract is the extract of guarana seeds as prepared in Example 2.

Moreover, “maintaining and/or increasing the concentration of healthy mammals” means maintaining and/or increasing the accuracy of analysis of information from an external stimulus chosen from a visual stimulus, an auditory stimulus, an olfactory stimulus or a gustatory stimulus with a view to decision-making in a healthy mammal. In an advantageous embodiment, it is an increase in accuracy in response to a visual stimulus, advantageously in healthy individuals. Thus, the improvement in concentration is a significant increase in the percentage of accuracy of a population of healthy individuals of at least 0.02%, advantageously of at least 0.05% after 30 days of supplementation measured within a population of healthy individuals administered the combination of the guarana extract and the extract of P. tricornutum according to the invention, compared with a population receiving a placebo ingredient. The measurement is performed within the framework of the clinical study described previously but via the Go-NoGo test (Donders, 1969; Wessel, 2017) which specifically assesses the ability to maintain sustained attention (concentration) and also the control of responses (decision-making) via the measurement of the accuracy of the response to visual stimuli by pressing a key representing “Go” or inhibiting a response by not pressing the key representing “No-Go”. This evaluation was performed with the same study population described previously and under strictly similar conditions. Even more advantageously, the combination administered is a combination of 500 mg of a guarana extract and a dose of 436 mg/day or 872 mg/day of a composition comprising the extract of P. tricornutum according to the invention, tocopherol and an MCT oil as described in the remainder of the present patent application, under the conditions as described in Example 4. Very advantageously, the extract of P. tricornutum included in the composition is the extract as prepared in Example 1 and the guarana extract is the extract of guarana seeds as prepared in Example 2.

In one embodiment of the invention, the extract of P. tricornutum is used on its own.

Advantageously, the extract is obtained by any extraction method known to those skilled in the art. Advantageously, the extract is obtained from the biomass of a microalgal culture. The biomass may first be centrifuged and then filtered to remove the water. A solid-liquid extraction step may then be performed. Preferentially, the extract is obtained by extraction in a solvent or solvent mixture, and advantageously in water, acetone, hexane, ethyl acetate, methyltetrahydrofuran, 2-methyloxolane, heptane, an alcohol chosen from ethanol, methanol, isopropanol, a natural or branched oil, a glycol, a polyol, a mixture of water/alcohol, water/glycol in a proportion of from 100/1 to 1/100 (w/w) or any other solvent making it possible to extract all or some of the compounds of hydrophobic and amphiphilic nature. Very preferentially, the extract of P. tricornutum is obtained by extraction in a water/ethanol mixture in a proportion of from 40/60 (w/w) to 1/100 (w/w), including 30/70 (w/w) and 20/80 (w/w). The solvent or mixture of solvents is separated from the residual biomass after extraction via processes such as centrifugation or filtration and may subsequently be concentrated, or the solvent removed, via techniques such as vacuum evaporation or any other technique allowing selective evaporation of the solvent in question. Alternatively, the extract of P. tricornutum according to the invention is obtained by extraction under subcritical or supercritical conditions. Advantageously in this case, the extract is obtained by supercritical CO₂ extraction. The extraction may be performed at a temperature ranging from 4° C. to 300° C., including room temperature, i.e. a temperature of 20° C. In a preferential embodiment of the invention, the extraction is performed at room temperature. In all cases, the extract obtained is filtered and optionally dried. Advantageously in this case, the drying step is performed by lyophilization, under vacuum drying, drum drying, or spray-drying, by fluidized bed with coupling by any technique allowing encapsulation or microencapsulation via a support matrix and/or the formation of an emulsion. The extract according to the invention is then in powder form. It may be incorporated into a composition alone or in combination as described hereinbelow. Alternatively, the extract of P. tricornutum is filtered but not dried and is in liquid form.

In another embodiment, the extract of P. tricornutum is used in combination with an extract chosen from a guarana (P. cupana) extract, an extract of ginseng (P. ginseng), an extract of G. biloba, an extract of N. nucifera, an extract of C. asiatica, an extract of P. nigrum, an extract of B. monnieri or any combination thereof and/or a compound chosen from creatine, arginine, caffeine, theanine, vitamin C, vitamin E, vitamin B, theophylline, paraxanthine, theobromine, rosmarinic acid, ellagic acid, cinnamaldehyde or any combination thereof.

In particular, the extract of P. tricornutum is used in combination with an extract chosen from a guarana (Paullinia cupana) extract, a Nelumbo nucifera extract, a Centella asiatica extract, a Piper nigrum extract, or any combination thereof and/or a compound chosen from creatine, arginine, caffeine, theophylline, paraxanthine, theobromine, rosmarinic acid, ellagic acid, cinnamaldehyde or any combination thereof.

Thus, an extract of guarana (P. cupana), ginseng (P. ginseng), G. biloba, N. nucifera, C. asiatica or P. nigrum may be obtained by extraction of all or part of the plant of interest chosen from the buds, the leaves, the stem, the flowers, the seeds or the rhizome. The extraction of guarana is advantageously an extraction of the seeds, the extraction of G. biloba is advantageously an extraction of the buds or the leaves, advantageously the leaves. The extraction of P. ginseng is advantageously an extraction of the rhizome, the extraction of N. nucifera is advantageously an extraction of the flowers and the extraction of P. nigrum is advantageously an extraction of the seeds.

The extraction may be performed by any method known to those skilled in the art chosen from maceration, hot decoction, grinding including ultrasonic grinding, using a blender, or else the extract may be obtained by extraction in water, notably under subcritical or supercritical conditions (carbon dioxide). The extraction may be performed using dry or fresh material, advantageously dry, in an amount of from 0.1% to 20% by weight, advantageously 1% to 20%, very advantageously 5% to 15%, and even more advantageously of 15% by weight relative to the total weight of the material and the extraction solvent. For the purposes of the invention, the term “dry matter” means dehydrated plant matter comprising less than 15%, advantageously less than 10%, even more advantageously less than 5% and very advantageously less than 1% water. The extraction may be performed at a temperature ranging from 4° C. to 300° C., including room temperature, i.e. a temperature of 20° C. In a preferential embodiment of the invention, the extraction will be performed at room temperature.

In one embodiment of the invention, the extraction is performed in water under subcritical conditions, at a temperature ranging from 100° C. to 300° C., advantageously from 120° C. to 250° C., even more advantageously at 120° C. The extraction may be performed at a given temperature or at successive increasing temperatures. In an advantageous embodiment of the invention, the extraction is performed at a temperature of 120° C. In an alternative embodiment, it may be performed with a gradient of three increasing temperatures of between 100° C. and 200° C., such as 120° C., 140° C. then 160° C. or 110° C., 130° C. then 150° C., or 120° C., 145° C. then 170° C.

Extraction under “subcritical conditions” means extraction in the presence of water, under conditions of temperature greater than 100° C. and pressure less than 221 bar, the water remaining in the liquid state but having a viscosity and surface tension lower than that of water at room temperature, increasing its dielectric constant. Thus, the extraction pressure is between 150 bar and 250 bar, preferentially between 200 bar and 221 bar, advantageously in a pressurized extraction autoclave.

In an alternative embodiment, the extract of guarana (P. cupana), ginseng (P. ginseng), G. biloba, N. nucifera, C. asiatica or P. nigrum may be obtained by extraction in a solvent or solvent mixture, and advantageously in water, acetone, hexane, ethyl acetate, methyltetrahydrofuran, 2-methyloxolane, heptane, an alcohol chosen from ethanol, methanol, isopropanol, a natural or branched oil, a glycol, a polyol, a water/alcohol or water/glycol mixture in a proportion of from 100/1 to 1/100 (w/w) or any other solvent which makes it possible to extract all or some of the compounds of hydrophobic and amphiphilic nature, such as ionic liquids, for example imidazolium or pyrrolidinium cations combined with chloride, acetate and tosylate anions. The extract obtained is then filtered and optionally dried by freeze-drying, vacuum drying, drum drying or spray-drying. The dried extract is thus in powder form. Alternatively, it may be in liquid form.

In an advantageous embodiment of the invention, the extract of P. tricornutum is used in combination with a guarana (Paullinia cupana) extract, a Nelumbo nucifera extract, a Centella asiatica extract, a Piper nigrum extract, or any combination thereof. In an advantageous embodiment of the invention, the extract of P. tricornutum is used in combination with a guarana extract, advantageously an extract of guarana seeds.

In yet another embodiment, the extract of P. tricornutum is used in combination with a compound chosen from creatine, arginine, caffeine, theanine, vitamin C, vitamin E, vitamin B, theophylline, paraxanthine, theobromine, rosmarinic acid, ellagic acid, cinnamaldehyde or any combination thereof, advantageously with a compound chosen from creatine, arginine, caffeine, theophylline, paraxanthine, theobromine, rosmarinic acid, ellagic acid, cinnamaldehyde or any combination thereof.

Caffeine (C₈H₁₀N₄O₂; molar mass: 194.19 g/mol, CAS No. 58-08-2), theophylline (C₇H₈N₄O₂; molar mass: 180.164 g/mol; CAS No.: 58-55-9), paraxanthine (C₅H₄N₄O₂; molar mass: CAS No: 69-89-6) and theanine (C₇H₁₄N₂O; molar mass: 174.2 g/mol, CAS No: 3081-61-6) may be obtained by extraction from any plant containing them, notably chosen from Coffea arabica, P. cupana, Camellia sinensis, Theobroma cacao. Theobromine (C₇H₈N₄O₂; molar mass: 180.164 g/mol, CAS No: 83-67-0) may be obtained by extraction of T. cacao. Creatine (C₄H₇N₃O; molar mass: 113.1 g/mol, CAS No: 60-27-5) and arginine (C₆H₁₄N₄O₂; molar mass: 174.2 g/mol, CAS No: 7200-25-1 or 157-06-2 for the D or R form) may be obtained commercially. Rosmarinic acid (C₁₈H₁₆O₈; molar mass: 360.31 g/mol, CAS No: 20283-92-5) may be extracted from the Salvia rosmarinus plant. Ellagic acid (C₁₄H₆O₈; molar mass: 302.19 g/mol, CAS No: 476-66-4) may be extracted from red fruits such as cherries, raspberries and blackberries. Cinnamaldehyde (C₉H₈O; molar mass: 132.16 g/mol, CAS No: 104-55-2) is the main component of cinnamon oil.

These compounds are extracted using a non-polar organic solvent or a mixture of non-polar organic solvents chosen from hexane, cyclohexane, heptane, trichloromethane, dichloromethane, acetone in a proportion of from 100/1 to 1/100 (w/w), 2-methyloxolane or ionic liquids, for instance imidazolium or pyrrolidinium cations combined with chloride, acetate and tosylate anions. They may be purified by preparative High Performance Liquid Chromatography (HPCL). Alternatively, they may be obtained commercially. Vitamin C (L-ascorbic acid, C₆H₈O₆, molar mass 176.12 g/mol, CAS No. 50-81-7) may be obtained commercially.

The use of the extract of P. tricornutum according to the invention, alone or in combination, is advantageously a use via the oral route. The extract, alone or in combination, may be included in a composition, advantageously for use as a food supplement.

The invention also relates to the combination of an extract of Phaeodactylum tricornutum and of an extract chosen from a guarana (P. cupana) extract, a ginseng (P. ginseng) extract, a G. biloba extract, an N. nucifera extract, a C. asiatica extract, a P. nigrum extract, a B. monnieri extract or a compound chosen from creatine, arginine, caffeine, theanine, vitamin C, vitamin E, vitamin B, theophylline, paraxanthine, theobromine, rosmarinic acid, ellagic acid, cinnamaldehyde and any combination thereof. In an advantageous embodiment of the invention, the invention also relates to the combination of an extract of Phaeodactylum tricornutum and of an extract chosen from a guarana (Paullinia cupana) extract, a Nelumbo nucifera extract, a Centella asiatica extract, a Piper nigrum extract, or a compound chosen from creatine, arginine, caffeine, theophylline, paraxanthine, theobromine, rosmarinic acid, ellagic acid, cinnamaldehyde and any combination thereof.

In one embodiment, the combination is characterized in that the weight ratio between the extract of P. tricornutum and the extract chosen from a guarana extract, a ginseng extract, a G. biloba extract, an N. nucifera extract, a C. asiatica extract, a B. monnieri extract or the compound is respectively from 1/6 to 6/1, advantageously from 2/1 to 4/1, even more advantageously from 3/1 to 7/2. This combination is synergistic. Very advantageously, according to the invention, it is a combination of an extract of P. tricornutum and a guarana (P. cupana) extract.

Preferentially, the guarana extract is a seed extract (P. cupana). Thus, a particularly advantageous combination comprises, by weight relative to the total weight of the combination:
an extract of P. tricornutum present in an amount of between 50 mg and 600 mg, advantageously from 220 mg to 440 mg,
a guarana (P. cupana) extract present in an amount of between 300 mg and 700 mg, advantageously 500 mg.

In an advantageous embodiment, the extract of P. tricornutum is prepared by solid-liquid extraction as already described in the invention. Preferentially, the extract of P. tricornutum is obtained by extraction in a water/ethanol mixture in a proportion of from 40/60 (w/w) to 1/100 (w/w), including 30/70 (w/w) and 20/80 (w/w), still preferentially in ethanol. Even more advantageously, the guarana extract (P. cupana) is obtained by hot aqueous-alcoholic extraction followed by concentration with evaporation under vacuum. The two extracts are filtered and then dried by freeze-drying, vacuum drying, drum drying or spray-drying. Each of the two extracts is in powder form and may be combined in the ratios described to obtain the combination.

The combination according to the invention is particularly useful for maintaining and/or increasing data processing speed and/or concentration in healthy mammals, advantageously healthy individuals not suffering from cognitive decline and/or disorder. Specifically, the applicant has unexpectedly demonstrated a synergistic effect of the combination of the extract of P. tricornutum and a guarana (P. cupana) extract in a weight ratio of respectively from 1/6 to 6/1, advantageously in a weight ratio of from 2/1 to 4/1 respectively, very advantageously in a weight ratio of from 3/1 to 7/2.

Thus, in one embodiment, “increasing data processing speed in healthy mammals” means herein significantly reducing the time required by at least 5 ms, advantageously by at least 10 ms, very advantageously by at least 20 ms to analyse information from an external stimulus chosen from a visual stimulus, an auditory stimulus, an olfactory stimulus or a gustatory stimulus with a view to making a decision, advantageously a visual stimulus, in a healthy mammal, advantageously a healthy individual.

Advantageously, this reduction in reaction time is measured within a population of healthy individuals to whom the combination according to the invention has been administered, compared with the reaction time measured within a population receiving a placebo ingredient. The measurement is advantageously performed within the framework of a clinical study using the Sternberg test (Sternberg S., 1969) as described previously. Four populations of healthy individuals are formed, the first receiving a placebo administration, the second receiving an administration of the combination of an extract of P. tricornutum and of a guarana extract, the third an administration of the extract of P. tricornutum alone and the fourth an administration of a guarana extract alone. Advantageously also, the combination is the combination of the extract of P. tricornutum and a guarana extract, advantageously a seed extract, in a weight ratio of respectively from 1/6 to 6/1, advantageously 2/1 to 4/1, very advantageously in a weight ratio of from 3/1 to 7/2.

Very advantageously, it is the extract of P. tricornutum as prepared according to Example 1 and the guarana extract is that as prepared in Example 2.

In an alternative embodiment, the combination of an extract of P. tricornutum and of a guarana (P. cupana) extract increases protein expression of a synaptotagmin chosen from synaptotagmin 1 (SYT1), SYT2, SYT3, SYT4, SYT5, SYT6, SYT7, SYT8, SYT9, SYT10, SYT11, SYT12, SYT13, SYT14, SYT15, SYT16, SYT17, in a neuron culture.

Advantageously, the increase in protein expression is at least 3%, advantageously at least 5%, of the protein expression of synaptotagmin in the neuronal culture compared with a control without said combination, whereas the extract of P. tricornutum alone increases said protein expression by only 2%. Similarly, the guarana extract alone only increases the synaptotagmin protein expression by 1% compared with the control without any guarana extract. In a particularly advantageous embodiment, it is an increase in the protein expression of synaptotagmin measured in the presence of the extract of P. tricornutum and a guarana (P. cupana) extract, advantageously a seed extract, in the weight ratio of from 2/1 to 4/1 respectively, very advantageously in the weight ratio of from 3/1 to 7/2. Even more advantageously, it is the extract of P. tricornutum as prepared according to Example 1 and the guarana extract is that as prepared in Example 2. In another alternative embodiment, the synergistic combination of an extract of P. tricornutum and a guarana (P. cupana) extract increases data processing speed in healthy mammals, advantageously healthy individuals, by increasing the length and/or dendricity of neuronal extensions in vitro. Thus, advantageously, the length of neuronal extensions is increased in an in vitro culture of neurons by at least 2.5%, preferentially by at least 5% in the presence of the combination of an extract of P. tricornutum and of a guarana (P. cupana) extract, compared with the increase in neuronal extensions measured in the presence of the extract of P. tricornutum alone or the guarana extract alone. In an advantageous embodiment, the synergistic combination is such that the weight ratio of the extract of P. tricornutum and guarana (P. cupana) extract is respectively from 1/6 to 6/1, advantageously 2/1 to 4/1 and very advantageously 3/1 to 7/2.

Very advantageously, it is the extract of P. tricornutum as prepared according to Example 1 and the guarana extract is that as prepared in Example 2.

In yet another alternative embodiment, the combination of an extract of P. tricornutum and of a guarana (P. cupana) extract increases data processing speed in healthy mammals, advantageously healthy individuals, by increasing in vitro the rate of calcium mobilization at synapses during synaptic transmission. An in vitro measurement of the level of calcium mobilized in a culture of neurons in the presence of the association or of each of the extracts characterizing it makes it possible to plausibly reflect an increase in data processing speed. In an advantageous embodiment, the measurement of the level of mobilized calcium is performed using a fluorescent probe which binds to calcium under the effect of its release following activation of neuronal receptors. Advantageously, the increase in data processing speed is measured in an in vitro culture of neurons in the presence of the combination of the extract of P. tricornutum and the guarana extract such that their weight ratio is respectively from 1/6 to 6/1, advantageously 2/1 to 4/1, very advantageously 3/1 to 7/2. Very advantageously, it is the extract of P. tricornutum as prepared according to Example 1 and the guarana extract is that as prepared in Example 2.

Another subject of the invention also relates to a composition comprising the combination according to the invention. In one embodiment, the composition is for use as a food supplement.

Thus, the composition comprises the combination of an extract of P. tricornutum and of an extract chosen from a guarana (P. cupana) extract, a ginseng (P. ginseng) extract, a G. biloba extract, an N. nucifera extract, a C. asiatica extract, a P. nigrum extract, a B. monnieri or any combination thereof and/or a compound chosen from creatine, arginine, caffeine, theanine, vitamin C, vitamin E, vitamin B, theophylline, paraxanthine, theobromine, rosmarinic acid, ellagic acid, cinnamaldehyde or any combination thereof, in particular such that the weight ratio between the extract of P. tricornutum and the chosen extract or compound is from 1/6 to 6/1, advantageously from 2/1 to 4/1, very advantageously from 3/1 to 7/2. Very advantageously, the composition comprises the combination of an extract of P. tricornutum and of an extract chosen from a guarana (Paullinia cupana) extract, a Nelumbo nucifera extract, a Centella asiatica extract, a Piper nigrum extract or any combination thereof, or a compound chosen from creatine, arginine, caffeine, theophylline, paraxanthine, theobromine, rosmarinic acid, ellagic acid, cinnamaldehyde or any combination thereof. Even more advantageously, the composition comprises the combination of an extract of P. tricornutum and of a guarana (P. cupana) extract, advantageously a seed extract, in particular in a weight ratio of respectively from 1/6 to 6/1, advantageously 2/1 to 4/1, very advantageously 3/1 to 7/2.

The composition may also include a plant oil and/or vitamin E. The term “plant oil” means any oil extracted from a plant or a seaweed, including a microalga, notably chosen from olive oil, rapeseed oil, linseed oil, sunflower oil and a medium chain triglyceride (MCT) oil. Medium Chain Triglycerides (MCT) are esters of glycerol and saturated fatty acids with a hydrocarbon-based chain of 6 to 12 carbon atoms. An MCT oil may thus be chosen from coconut kernel oil, advantageously coconut oil, palm kernel oil and palm oil, but may also be obtained from other fats or oils. Advantageously for the purposes of the invention, the plant oil is an MCT oil, more advantageously coconut kernel oil, very advantageously coconut oil. In a particularly advantageous embodiment of the invention, the plant oil is present in an amount of from 20% to 80%, advantageously still from 30% to 60%, by weight relative to the total weight of the composition. The term “vitamin E” means a tocopherol chosen from α-tocopherol, γ-tocopherol, β-tocopherol or 6-tocopherol, or a tocotrienol chosen from α-tocotrienol, β-tocotrienol, γ-tocotrienol or 6-tocotrienol. Advantageously, it is α-tocopherol. In an advantageous embodiment, vitamin E is present in the composition in an amount by weight relative to the total weight of the composition of from 0.15% to 1.25%, advantageously from 0.25% to 1%, very advantageously of 0.5%. Thus, an advantageous composition of the invention comprises at least, by weight relative to the total weight of the composition:
an extract of P. tricornutum in an amount of from 10% to 25%, advantageously 11% to 16%,
an MCT oil present in an amount of from 30% to 60%,
α-tocopherol present in an amount of from 0.25% to 1%, advantageously 0.5%,
caffeine or theine in an amount of from 5% to 25%, advantageously 12%.

Another particularly advantageous composition comprises at least, by weight relative to the total weight of the composition:
an extract of P. tricornutum in an amount of from 10% to 25%, advantageously 11% to 16%,
an MCT oil present in an amount of from 30% to 60%,
α-tocopherol present in an amount of from 0.25% to 1%, advantageously 0.5%,
arginine in amounts of from 5% to 25%.

Another advantageous composition further comprises at least, by weight relative to the total weight of the composition:
an extract of P. tricornutum in an amount of from 10% to 25%, advantageously 11% to 16%,
an MCT oil present in an amount of from 30% to 60%,
α-tocopherol present in an amount of 0.5%,
a guarana (P. cupana) extract in an amount of from 35% to 55%, advantageously an extract of guarana (P. cupana) seeds.

Moreover, the composition of the invention comprises at least one additive chosen from preserving agents, colourings, flavourings, disintegrants, lubricants and coating or encapsulating agents. The composition is in the form of gel capsules, wafer capsules, tablets, pellets, liquid or loose powder. It is advantageously packaged in doses with a unit weight of between 11 mg and 2 g, advantageously between 20 mg and 2 g. When in liquid form, the composition may be incorporated into an energy drink, fruit juice, syrup or water, or may be formulated in the form of “soft gel” capsules.

It is used as a food supplement, particularly for sportspeople, video gamers and students. In this case, the composition is administered orally in a dose of between 1 g and 2 g for a period of at least 1 day, advantageously at least 15 days, very advantageously 1 month. Alternatively, the composition is used as a food supplement in animals, advantageously domestic animals, even more advantageously dogs and cats, very advantageously dogs. In this case, the composition is administered orally alone or mixed with a foodstuff, at a daily dose of between 11 mg and 25 mg per kg of body mass.

The composition according to the invention is used for maintaining and/or increasing data processing speed and/or concentration in healthy mammals, advantageously healthy individuals.

Examples referring to the description are given hereinbelow. These examples are illustrative and are not intended to limit the scope of the invention. The examples form an integral part of the present invention and any new feature relative to a prior art from the description taken as a whole, forms an integral part of the invention.

Unless otherwise stated, the percentages are given in weight/weight, the temperature is given in degrees Celsius and the time is given in milliseconds (ms).

### LIST OF FIGURES

FIG. 1 represents the results of the Sternberg Test performed according to the clinical study (Example 3): effect of a combination of the extract of P. tricornutum and of a guarana (P. cupana) extract on the reduction and/or maintenance of the reaction time during the study and as a function of the level of difficulty presented (number of visual stimuli d) in healthy individuals. The data are means and 95% confidence intervals. Changes relative to baseline are indicated by † (p<0.05 change relative to baseline) and ‡ (p>0.05 to p<0.10 trends relative to baseline). Lower case letters indicate p<0.05 differences relative to placebo (p), low dose (I) or high dose (h), while upper case letters (P, L, H) indicate trends (p>0.05 to p<0.10).

FIG. 2 represents the results of the Go-NoGo Test performed according to the clinical study (Example 4): effect of a combination of the extract of P. tricornutum and of a guarana (P. cupana) extract on maintaining and increasing concentration in healthy individuals. The data are means and 95% confidence intervals. Changes relative to baseline are indicated by † (p<0.05 change relative to baseline) and ‡ (p>0.05 to p<0.10 trends relative to baseline). Lower case letters indicate p<0.05 differences relative to placebo (p), low dose (1) or high dose (h), while upper case letters (P, L, H) indicate trends (p>0.05 to p<0.10).

### EXAMPLES

### Example 1: Method for Preparing an Extract of Phaeodactylum tricornutum

The extract of the microalga P. tricornutum is obtained from the biomass of a microalgal culture from a strain originating in France. The biomass is first centrifuged and then filtered to remove the water. A solid-liquid extraction step is then performed.

The extract is thus obtained by extraction in a water/ethanol mixture in a proportion of from 40/60 (w/w) to 1/100 (w/w), at a temperature of 20° C., i.e. at room temperature, for a period of 2 hours. It is then filtered and dried by freeze-drying. It is in the form of a powder.

### Example 2: Method for preparing a guarana (Paullina cupana) extract and combination of an extract of Phaeodactylum tricornutum and of a guarana (P. cupana) extract

### 2.a): Preparation of a Quarana Extract:

The guarana extract is obtained by extracting the seeds of the Paullinia cupana plant, which originated in France, in a water/ethanol mixture (20/80; v/v). Extraction is performed at a temperature of 80° C. for a period of 2 hours.

The extract is then filtered and dried by freeze-drying. It is in the form of a powder.

### 2.b): Commercial Quarana Extract

The aqueous-alcoholic extract (water/ethanol) of the seeds of the Paulinia cupana (guarana) plant may be purchased from the company Natac or the company Nexira. It is in powder form.

2.c): Preparation of the Combination of Phaeodactylum tricornutum Extract and Quarana (P. cupana) Extract

The guarana extract obtained according to Example 2.a) or 2.b) may be mixed with the powder obtained in Example 1 to obtain the combination according to the invention in the respective proportions by weight of P. tricornutum extract and guarana of 2/9 to 3/7 (Example 2c1) or 3/1 to 7/2 (Example 2c2).

### Example 3: Effect of an Extract of Phaeodactylum tricornutum on Maintaining and/or Increasing Data Processing Speed and/or Concentration in Healthy Individuals

Protocol: A double-blind, placebo-controlled clinical trial was conducted on a population of 51 men and 10 women recruited as experienced video game players (mean age 21.7±4 years, mean weight 73.0±13 kg, mean mass distribution 24.2±3.6 kg/m²). The population of interest was randomly divided into three groups and administered supplements as follows:
Control group: two placebo gel capsules of 436 mg/day (436 mg of sunflower oil in a capsule resembling a capsule containing the extract of P. tricornutum) and one capsule of 500 mg/day containing microcellulose (resembling a guarana capsule).
Treatment 1—known as low dose: combination of one capsule comprising the extract of P. tricornutum as prepared according to Example 1 (dose: 436 mg), α-tocopherol and an MCT oil, one placebo capsule of 436 mg/day and one capsule of guarana extract of 500 mg/day.
Treatment 2—known as high dose: combination of two capsules, each containing the extract of P. tricornutum as prepared according to Example 1 (dose: 436 mg), α-tocopherol and an MCT oil, and one capsule of guarana extract (500 mg/day). The supplements were prepared to maintain double-blind administration throughout the study. The inclusion criteria were as follows:
1.) men and women in good health;
2.) between the ages of 18 and 40;
3.) self-reported history of playing video games for 5 hours or more per week in the 6 months prior to screening;
4.) body Mass Index (BMI) of between 18 and 34.9 kg/m²;
5.) subjects agreed to provide their own operator-oriented action or strategy video game, which they had played at least 21 times in the preceding 3 months prior to the start of the clinical study;
6.) no recent ingestion (<2 weeks) of food supplements affecting cognitive function;
7.) be able to give written informed consent and consume the experimental product on a daily basis for the duration of the study;
8.) to live freely (to live in a private home, alone or with family, and to be able to maintain their health and hygiene without assistance);
9.) be willing to maintain a constant sleep duration on the evening before the study visits;
10.) agree to continue their usual use of the game between study visits.

The criteria for non-inclusion were as follows (the participants were not allowed to take part in the study in the following cases):
1.) caffeine and alcohol consumption during the 12 hours preceding each study visit;
2.) consumption of food supplements likely to affect cognition and/or having a stimulating effect at least 7 days before Visit 2;
3.) pregnant women, breast-feeding women or women wishing to become pregnant;
4.) presence of untreated major psychotic or depressive disorder or any history of cognitive impairment;
5.) hypertension, diabetes, thyroid disease, uncontrolled heart disease, cancer;
6.) significant neurological disease;
7.) anticipated major changes in lifestyle (diet, level of exercise, travel) during the study period;
8.) history of alcohol or drug abuse in the last 12 months;
9.) known allergy to one of the ingredients of the supplementation product;
10.) individuals unwilling to provide their own game system and/or own game.

During a familiarization session (Visit 1), participants were informed about the study and signed an informed consent form. The participants answered questionnaires about their medical history, underwent a general physical examination including determination of height, weight, resting heart rate and blood pressure, and were briefed on the general study procedures. Those eligible to participate were scheduled to complete the baseline assessments and practice the study assessments. The participants were asked to record their food and fluid intake for 4 days prior to the test, to refrain from consuming atypical amounts of caffeine and other stimulants not normally consumed in their diet for 48 hours, and to fast for 12 hours prior to the test. During this visit, the classification of the video game (action or strategy oriented) was confirmed. Each subject was asked to bring along the chosen, pre-approved video game, and also a compatible gaming platform and accessories. Subjects had to have played the chosen game at least 21 times in the 3 months prior to selection. Subjects agreed to bring and play the same video game at Visit 1 and Visit 2 and to play the chosen game regularly, without excess between study visits, in order to minimize changes in learning curve bias. The last score of the chosen game before dosing was recorded. It was provided in the form of a photo or screenshot, but not in the form of a verbal response.

Experiment 1 (Visit 2): The participants reported to the laboratory and handed in a 4-day food diary, were weighed, completed questionnaires on stimulant sensitivity and side effects, and performed the Go-NoGo Sternberg tests. They were then randomly assigned to ingest the supplements as described. Fifteen minutes after ingestion, the participants repeated the tests (Post-15-SUPP). The time between ingestion of the treatment and the start of the video game was recorded. The participants then played their video game for 1 hour. Immediately afterwards, the participants performed the battery of tests again, and also a test on stimulants and side effects. The participants then left the laboratory and received the appropriate amount of the assigned treatment with instructions on how to take it (Post-Gaming).

Experiment 2 (Visit 3): After 4 weeks of daily supplementation, participants reported to the laboratory and handed in 4-day food diaries, were weighed and performed the same battery of tests as previously (Experiment 1) (Pre-SUPP). They then ingested the treatment assigned to the study and/or the placebo just after the pre-game tests and waited the same length of time as recorded in Experiment 1. The participants then played their video game (same video game as in Experiment 1) for 1 hour. Immediately afterwards, the participants repeated the battery of tests and completed questionnaires on stimulant sensitivity and side effects, and also a test on stimulants (Post-Gaming). This study was conducted in accordance with the guidelines and principles of the Food and Drug Administration (FDA), the International Conference on Harmonization (ICH) and Good Clinical Practice (GCP).

The subjects were instructed to take their dedicated supplement once daily for 1 month (±2 days). The first and last doses were administered at the clinical site during Visit 2 (Experiment 1) and Visit 3 (Experiment 2), respectively.

Data were analysed using univariate, multivariate and repeated-measure general linear models (GLM), taking into account the respective baseline characteristics as covariates where required. Furthermore, changes relative to baseline were assessed by means with 95% confidence intervals and analysed by one-way ANOVA. At the end of the tests, a power analysis was also performed.

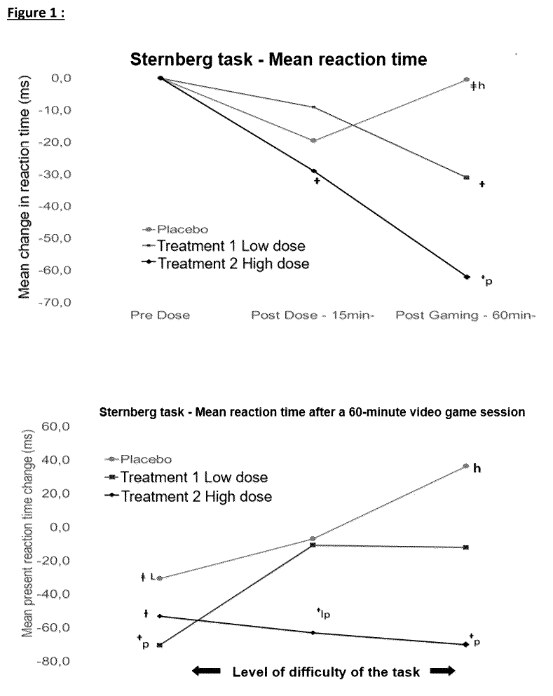

Results: The results of the Sternberg test are presented in the two tables below (the corresponding graphs are shown in the corresponding FIG. 1) and correspond to the results of the first experiment (1 single supplementation received).

TABLE 1
Average reduction in reaction time in the Sternberg test
in the three populations that received the supplement
(Placebo, Treatment 1, Treatment 2) (FIG. 1 left)

| | | Treatment 1 - | Treatment 2 - |
|---|---|---|---|
| | Placebo | Low dose | High dose |
| Pre-Dose | 0.00 | | |
| Post Dose - 15 min - | −19.55 | −9.02 | −28.99 |
| Post gaming - 60 min- | −0.45 | −31.08 | −62.03 |

TABLE 2
Average reduction or maintenance of reaction time in the Sternberg
test in the three populations that received the supplement
(Placebo, treatment 1, treatment 2) as a function of the number
of stimuli (data to be processed d) reflecting an increasing
level of difficulty after a game session (FIG. 1 right).

| | | Level of | Level of |
|---|---|---|---|
| | Level of | difficulty | difficulty |
| | difficulty d = 2 | d = 4 | d = 6 |
| Placebo | −30.69 | −6.98 | 36.32 |
| Treatment 1 - Low dose | −70.40 | −10.86 | −12.14 |
| Treatment 2 - High dose | −53.10 | −62.94 | −70.05 |

Conclusion: The results showed an effect of the combination of guarana and of the extract of P. tricornutum compared with the placebo ingredient, with a predominant effect of the extract of P. tricornutum within this combination, on the increase in data processing speed in healthy individuals, this effect being characterized by both:
a shorter reaction time for individuals receiving a low or high dose of the composition in response to the visual stimulus relative to individuals in the placebo group;
a greater reduction in reaction time due to the composition comprising the extract of P. tricornutum at a high dose (872 mg) than at a low dose (436 mg), reflecting a greater benefit from the extract of P. tricornutum;
this reaction time remained constant when the number of visual stimuli d (data to be analysed) increased. It should be noted that the changes in reaction times associated with supplementation as a function of the number of visual stimuli (n), are the opposite of the results commonly obtained in the Sternberg test. Indeed, when the number of incoming stimuli (the amount of data to be evaluated before making a decision) increases, the reaction time increases mechanically because data processing is serial and exhaustive (Sternberg, 1966), and each individual has a constant native processing time per unit of data. Supplementation with a combination of low-dose extract of P. tricornutum (436 mg) and guarana extract induced a constant reaction time from a number of stimuli of 4 to 6. Supplementation with a combination of high-dose extract of P. tricornutum (872 mg) and guarana extract, on the other hand, induced a decreasing reaction time from 2 stimuli up to 6.

### Example 4: Effect of an Extract of Phaeodactylum tricornutum on Maintaining and/or Increasing Concentration in Healthy Individuals

Protocol: the protocol is that shown in Example 3.

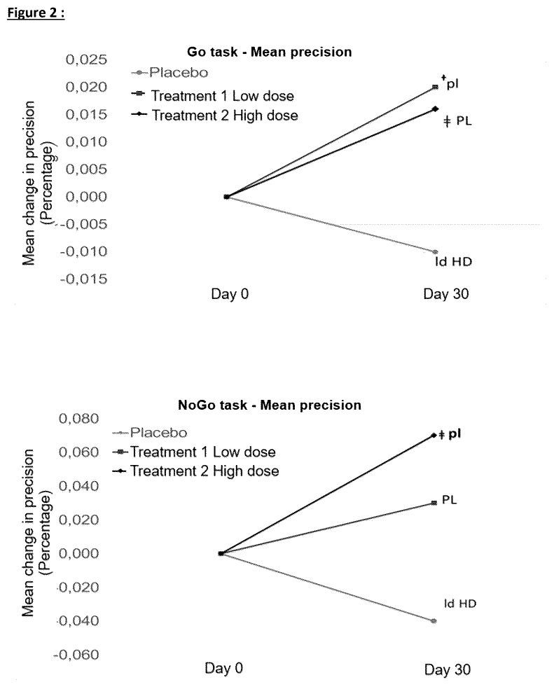

Result: The results are shown in Table 3 (FIG. 2) (Go-NoGo test).

TABLE 3
Average increase (±standard deviation) representing
the percentage change in the level of concentration on
Go-NoGo tasks after 30 days of supplementation (FIG. 2)

| | | Treatment 1 - | Treatment 2 - |
|---|---|---|---|
| | Placebo | Low dose | High dose |
| Day 0 | 0.00 | | |
| Day 30 -Go test- | −0.01 | 0.02 | −0.02 |
| Day 30 - NoGo test- | −0.04 | 0.03 | 0.07 |

Conclusion: Supplementation with the combination of guarana extract and the composition containing the extract of P. tricornutum improved the level of precision of the responses given after 30 days of supplementation, regardless of the dose (low or high) of the composition containing the extract of P. tricornutum, whereas the level of precision decreased in the placebo group (FIG. 2). The benefits in terms of concentration level were greater with the composition containing the high-dose extract of P. tricornutum (FIG. 2 right).

### Example 5: In Vitro Effect of a Synergistic Combination of an Extract of P. tricornutum and a Guarana (P. Cupana) Extract on Maintaining and/or increasing data processing speed in healthy individuals

### Example 5a): Increase in the Length and Dendricity of Neuronal Extensions

Sensory neurons were obtained from hiPS cells (human induced pluripotent stem cells), which were themselves obtained from human fibroblasts from a healthy donor. The cells were seeded in 96-well plates and maintained for 6 days in culture in a differentiation-inducing medium at a temperature of 37° C. (5% CO₂). The culture medium was changed every 2 days. After a period of 8 days in culture, the culture medium was changed for a maturation medium (medium with growth factors including Nerve Growth Factor). The cells were maintained in culture at a temperature of 37° C. under 5% CO₂.

After 1 day of induction, the growth factors in the culture medium were removed and replaced with a control comprising NGF growth factor only; or the extract of P. tricornutum according to Example 1; or the guarana extract according to Examples 2a) or 2b); or the combination according to Example 2c) in the respective P. tricornutum/guarana weight ratios of 2/9 to 3/7 (Example 2c1) or 3/1 to 7/2 (Example 2c2).

The media were cultured for a period of 4 days and the cells were then recovered and labelled with an anti-β-tubulin antibody and then visualized using a fluorochrome-coupled secondary antibody (labelling of the structure of the neurons i.e. cell body and neurites). The length of neuronal extensions and the number of bifurcations at the level of these extensions were measured using an algorithm that automatically detected neurites and their bifurcations on 20 images acquired at x20 for each culture well. An average of 6 wells was established for each condition, compared with the control and a statistical analysis was then performed (One Way Anova with Dunnett correction).

### Example 5b): In Vitro Increase in the Rate of Calcium Mobilization at Synaptic Level

Sensory neuron cells as described in Example 5a) were seeded in 96-well plates and maintained for a period of 6 days in culture in a differentiation-inducing medium at a temperature of 37° C. (5% CO₂). The culture medium was changed every 2 days. After a period of 9 days in culture, the culture medium was changed to a maturation medium (medium with growth factors including Nerve Growth Factor) and the extract of P. tricornutum according to Example 1; or the guarana extract according to Example 2a) or 2b); or the combination of the two extracts according to Example 2c) at the respective P. tricornutum/guarana weight ratios of 2/9 to 3/7 (Example 2c1) or 3/1 to 7/2 (Example 2c2); or a control (NGF growth factor) were added to the culture medium. The cells were maintained in culture at a temperature of 37° C. under 5% CO₂ for a period of 19 days, after which time the neuronal receptors become functional. The TRPM8 (Transient receptor potential cation channel subfamily M (melastatin) member) neuronal receptors were specifically inhibited and the media incubated with a fluorescent probe (Fluo-4-AM; ThermoFisher) in order to specifically activate these receptors. An increase in fluorescence reflected activation of the receptors, and therefore a massive influx of calcium into the neurons, reflecting an increase in data processing speed in healthy individuals. Fluorescence variations were recorded over a period of 2 minutes and the average for each condition was calculated (n=6). Statistical analysis was performed (One way Anova).

### REFERENCES

Donders, F. C. (1969). On the speed of mental processes. Acta Psychologica, 30, 412-431.
Sternberg S. (1966) High-speed scanning in human memory. Science. 153(3736): 652-4.
Wessel, J. R. (2017). Prepotent motor activity and inhibitory control demands in different variants of the go/no-go paradigm. Psychophysiology. doi: 10.1111/psyp.12871

1-15. (canceled)

16. A method for maintaining and/or increasing data processing speed and/or concentration in healthy individuals not suffering from cognitive decline and/or disorder, the method comprising the administration to an individual in need thereof/who desires so of an effective amount of an extract of Phaeodactylum tricornutum alone or in combination with an extract chosen from a guarana (Paullinia cupana) extract, a Nelumbo nucifera extract, a Centella asiatica extract, a Piper nigrum extract, or any combination thereof and/or a compound chosen from creatine, arginine, caffeine, theophylline, paraxanthine, theobromine, rosmarinic acid, ellagic acid, cinnamaldehyde or any combination thereof.

17. The method according to claim 16, wherein the extract of Phaeodactylum tricornutum is obtained by extraction in water, acetone, hexane, ethyl acetate, methyltetrahydrofuran, 2-methyloxolane, heptane, an alcohol chosen from ethanol, methanol, isopropanol, a natural or branched oil, a glycol, a polyol, a water/alcohol or water/glycol mixture in a proportion of from 100/1 to 1/100 (w/w).

18. The method according to claim 16 wherein the administration is an oral administration.

19. The method according to claim 18 wherein the extract or the combination is in the form of a food supplement.

20. Combination of an extract of Phaeodactylum tricornutum and an extract chosen from a guarana (Paullinia cupana) extract, a Nelumbo nucifera extract, a Centella asiatica extract, a Piper nigrum extract, or a compound chosen from creatine, arginine, caffeine, theophylline, paraxanthine, theobromine, rosmarinic acid, ellagic acid, cinnamaldehyde and any combination thereof.

21. Combination according to claim 20, wherein the weight ratio between the extract of Phaeodactylum tricornutum and the extract or the compound is from 1/6 to 6/1.

22. Combination according to claim 20, wherein the extract is a guarana (Paullinia cupana) extract.

23. Combination according to claim 22, which contains:
an extract of P. tricornutum present in an amount of between 50 mg and 600 mg,
a guarana (P. cupana) extract present in an amount of between 300 mg and 700 mg.

24. Composition comprising the combination according to claim 20, a plant oil and vitamin E, wherein the plant oil is present in the composition in a concentration of 30% to 60% by weight and the vitamin E is present in a concentration of 0.25% to 1% by weight relative to the total weight of the composition.

25. Composition according to claim 24, which comprises:
an extract of P. tricornutum in an amount of from 10% to 25%,
an MCT oil present in an amount of from 30% to 60%,
α-tocopherol present in an amount of 0.5%,
a guarana (P. cupana) extract in an amount of from 35% to 55%.

26. Composition according to claim 24, which also comprises at least one additive chosen from preserving agents, colourings, flavourings, disintegrants, lubricants and coating or encapsulating agents.

27. Composition according to claim 24, which is in the form of gel capsules, wafer capsules, tablets, pellets, liquid or loose powder.

28. Composition according to claim 24, which is packaged in doses having a unit weight of between 20 mg and 2 g.

29. Composition according to claim 24, which is administered at a dose of between 1 g and 2 g over a period of at least 1 day.

30. A method for maintaining and/or increasing data processing speed and/or concentration in healthy individuals not suffering from cognitive decline and/or disorder, the method comprising the administration to an individual in need thereof/who desires so of an effective amount of the combination according to claim 20, or of a composition comprising the combination according to claim 20, a plant oil and vitamin E, wherein the plant oil is present in the composition in a concentration of 30% to 60% by weight and the vitamin E is present in a concentration of 0.25% to 1% by weight relative to the total weight of the composition

31. The method according to claim 16, wherein the extract of Phaeodactylum tricornutum is obtained by extraction in a water/ethanol mixture in a proportion of from 40/60 (w/w) to 1/100 (w/w).

32. Combination according to claim 22, wherein the guarana (Paullinia cupana) extract is a seed extract.

33. Combination according to claim 22, which contains:
an extract of P. tricornutum present in an amount of from 220 mg to 440 mg,
a guarana (P. cupana) extract present in an amount of 500 mg.

34. Composition according to claim 24 which is a food supplement.

35. Composition according to claim 24, which comprises:
an extract of P. tricornutum in an amount of from 11% to 16%,
an MCT oil present in an amount of from 30% to 60%,
α-tocopherol present in an amount of 0.5%,
a guarana (P. cupana) seed extract in an amount of from 35% to 55%.
